(19) United States
(12) Patent Application Publication
(10) Pub. No.: US 2009/0246270 A1
(43) Pub. Date: Oct. 1, 2009

(54) Use of Iontophoresis or Electrotherapy and Ultrasound to Deliver Agents for Skin Rejuvenation.

(76) Inventor: Dennis R. Jones, Chesapeake, VA (US)

Correspondence Address: Dennis R. Jones;Ste:112, 1025 Executive Blvd., Chesapeake, VA, 23320, US

(21) Appl. No.: 12/360,176

(22) Filed: Jan. 27, 2009

Related U.S. Application Data
(60) Provisional application No. 61/025,182, filed on Jan. 31, 2008.

(51) Int. Cl. A61K 9/127

(52) U.S. Cl. 424/450

(57) ABSTRACT

The method disclosed includes the combining of iontophoresis or electrotherapy and ultrasound to deliver the appropriate chromophores to the skin for cosmetic or therapeutic treatment of the skin and skin conditions by the use of a laser, Intense Pulsed Light (IPL), a combination of laser and Radio Frequency (RF) energy, or a combination of IPL and RF.

This application claims priority to Provisional Application No. 61/025,182. The filing date of provisional application No. 61/025,182 is Jan. 31, 2008 and there is no new matter in this application.

### FIELD OF INVENTION

The method disclosed includes the combining of iontophoresis or electrotherapy and ultrasound to deliver the appropriate chromophores to the skin for cosmetic or therapeutic treatment of the skin and skin conditions by the use of a laser, Intense Pulsed Light (IPL), a combination of laser and Radio Frequency (RF) energy, or a combination of IPL and RF.

The treatments include but are not limited to: skin rejuvenation, collagen remodeling, the reduction or removal of fine lines wrinkles, skin tightening, skin lifting, shrinking the skin following liposuction, treatment of acne, treatment of melasma, sun spots, skin cancer, removal of moles, removal of skin tags, removal of warts, reduction of odor production.

### BACKGROUND OF INVENTION

Historically, there have been a number of options for cosmetic or therapeutic treatment of the skin by the use of a laser, Intense Pulsed Light (IPL), a combination of laser and Radio Frequency (RF) energy, or a combination of IPL and RF. However, the treatments include the use of a light source that targets a chtomophore residing in the skin.

Problems exist with the current techniques, however. The success for the treatments listed above is in proportion to the availability of the targeted chromophore residing in the skin, which can vary greatly from patient to patient.

The use of a light sources as a method of treating various pathological and cosmetic conditions, particularly skin rejuvenation, can be divided into two general methods. The first being essentially the application of laser or lightsource energy selected to be absorbed preferentially by elements residing naturally within the skin. The second being differentiated from the first by introduction of a foreign element to provide an effective radiation target with an absorption frequency equal to or different from the naturally occurring elements in the skin.

### SUMMARY OF THE INVENTION

This application claims priority to Provisional Application No. 61/025,182. The filing date of provisional application No. 61/025,182 is Jan. 31, 2008 and there is no new matter.

It is an objective of the present disclosure to provide an improved method of: skin rejuvenation, collagen remodeling, the reduction or removal of fine lines wrinkles, skin tightening, skin lifting, shrinking the skin following liposuction, treatment of acne, treatment of melasma, reduction or removal of sun spots, treatment of skin cancer, removal of moles, removal of skin tags, removal of warts, reduction of odor production. This new method employs iontophoresis or electrotherapy and ultrasound or High Intensity Focused Ultrasound (HIFU) simultaneously or in conjunction with transdermal delivery of agents that may be delivered in pure form, in solution, as emulsions, within liposomes, within synthetic or natural microspheres, polymeric nanoparticles, microparticles, microcapsules, or other known micro encapsulation vehicles. Agents may include, without limitation, natural or synthetic melanin, aminolevulinic acid (ALA), hair dyes, vegetable dyes, food coloring, fabric dyes, tissue stains, shoe or leather dyes, and other plant products such as flavonols, cholorophyll, caroteniods, enzymes, etc.

Agents may be delivered in pure form, in solution, in emulsions, in liposomes, in synthetic or natural microspheres, polymeric nanoparticles, micropaiticles, microcapsules, or other known micro encapsulation and include a topical gel, lotion, liquid, cream or ointment composition that may also include melanin encapsulated liposomes. The agent is designed to attach to, or become physically incorporated into, the skin, the hair follicle, the hair bulb, the hair duct and or papilla.

The ultrasound energy causes break up of the top most surface of the skin and allows the composition to migrate into the skin. This technique preferentially ultra-exposes the skin to light energy which is absorbed preferentially by naturally occurring and synthetic elements in skin. Melanin is one of the most common chromophores found in the hair follicle used for the selective photothermolysis of unwanted hair, and other chromophores such as ALA can be used also. Laser or other intense pulsed light between 100 and 3000 nm is appropriate for targeting melanin and ALA in the hair shaft.

The combined use of melanin encapsulated liposomes or other chromophores in conjunction with iontophoresis or electrotherapy and ultrasound or iontophoresis or electrotherapy and HIFU followed by a treatment using a laser, IPL, a combination of laser and RF energy, or a combination of IPL and RF allows for immediate skin rejuvenation in selected locations on the body of mammals. This combination is so effective that the patient can be treated with a single or at least minimal office visitation.

It is another object of the disclosure to provide a composition where the liposomes have a size range of 1 nanometer (nm) to 500 microns. However, a diameter between manometer and 10 microns is preferred.

It is another object of the disclosure to provide a method of hair removal which increases the quantity of melanin or other chromophores surrounding the hair follicle to create an enhanced target for incident light energy and improve the preferential absorption in the proximity of the hair follicle.

It is also an object of the disclosure to provide a method of destroying skin cancer cells by photothermolysis by selective delivery of melanin using liposomes to create an enhanced target for subsequent application of light energy, which minimizes required irradiation and thereby minimizes the damage to the surrounding skin.

It is a further object of the disclosure that a variety light energy sources, both coherent and incoherent, including such lasers as ruby red and Nd-YAG, alexandrite, and diode lasers, or any other light source providing an effective frequency and intensity.

It is also another object of the disclosure that the light energy should be in the wavelength range of 100 to 3000 nm.

It is also another object of the disclosure that the pulse length is from about 1 nanoseconds to about 1000 milliseconds.

The combined use of iontophoresis or electrotherapy and ultrasound, the appropriate chromophore and laser light allows for immediate skin rejuvenations in selected locations on the body of mammals. This combination is so effective that the patient can be treated with a single or at least minimal office visitations. Thereby eliminating multiple applications of products intended to achieve the same results.

1. A method of providing permanent skin rejuvenation employing a combination of; iontophoresis or electrotherapy and ultrasound, IPL and laser or equivalent light energy, wherein said combination includes using iontophoresis or electrotherapy and ultrasound or HIFU simultaneously for transdermally delivering natural or synthetically derived melanin, ALA, hair dyes, vegetable dyes, food coloring, fabric dyes, tissue stains, shoe leather dyes, and other plant derived products optionally including; flavanols, chlorophyll, carotenoids, and enzymes, by using a topical gel, lotion, liquid, cream or ointment formulation, said method also comprising melanin encapsulated liposomes using ultrasound for causing break up of the top most surface of the skin allowing said melanin encapsulated liposomes composition or other chromophores to migrate into said skin and hair follicles such that said liposome composition or other chromophores may reside in or become physically incorporated into the skin, the hair shaft, the hair follicles, the hair bulb, or the hair duct so that using IPL, laser, a combination of laser and RF, a combination of IPL and RF or equivalent light energy results in preferential ultra-exposure of said skin providing focus for the absorption of heat that produces skin rejuvenation.

2. The method of claim 1, wherein said permanent skin rejuvenation and treatments listed can be provided in one or more patient treatments.

3. The method of claim 1, wherein said permanent skin rejuvenation is provided in a single treatment.

4. The method of claim 1, wherein melanin in the skin is the chromophore for treatments (listed above).

5. The method of claim 1, wherein a laser or other intense pulsed light between 300 nm to 3000 nm is appropriate for targeting melanin in the skin.

6. The method of claim 1, wherein removal employing a combination of; iontophoresis or electrotherapy, ultrasound or HIFU, and laser or equivalent light energy eliminates the need to continuously prepare and re-prepare the skin with melanin or other chromophores prior to multiple in office laser treatments for completing desired skin rejuvenation or treatments listed.

7. The method of claim 1, comprising the step of exposing the skin to at least one enzyme prior to exposure to ultrasound and iontophoresis or electrotherapy.

8. The method of claim 1, wherein said pulse length for laser light is from about 1 nanosecond to about 1 second.

9. The method of claim 8, wherein said pulse length is from about 1 nanoseconds to about 1000 milliseconds.

10. The method according to claim 1, wherein the laser or equivalent light energy fluence received at said hair producing tissue is 300 J/cm² or less.

11. The method according to claim 10, wherein said energy fluence received at said tissue is from about from about 1×10⁻⁶ J/cm² to 1 J/cm².

12. The method of claim 11, wherein said energy fluence received at said tissue is from about from about 1×10⁻³ J/cm² to about 0.1 J/cm².

13. The method comprising cooling said tissue to maintain a temperature of said tissue below a threshold for thermal injury.

14. The method of claim 13, further comprising the step of maintaining the temperature of said tissue at or below 38 degrees Celsius.

15. A set of devices for providing skin rejuvenation and said treatments employing a combination of; iontophoresis or electrotherapy and ultrasound, IPL and laser or equivalent light energy, wherein said combination includes using iontophoresis or electrotherapy and ultrasound or HIFU simultaneously for transdermally delivering natural or synthetically derived melanin, ALA, hair dyes, vegetable dyes, food coloring, fabric dyes, tissue stains, shoe leather dyes, and other plant derived products optionally including; flavanols, chlorophyll, carotenoids, and enzymes, by using a topical gel, lotion, liquid, cream or ointment formulation, said method also comprising melanin encapsulated liposomes using ultrasound for causing break up of the top most surface of the skin allowing said melanin encapsulated liposomes composition or other chromophores to migrate into said skin and hair follicles such that said liposome composition or other chromophores may reside in or become physically incorporated into the skin, the hair shaft, the hair follicles, the hair bulb, or the hair duct so that using IPL, laser, a combination of laser and RF, a combination of IPL and RE or equivalent light energy devices results in preferential ultra-exposure of said skin providing focus for skin rejuvenation and said treatments.

16. The devices of claim 15, wherein said skin rejuvenation and said treatments can be provided in one or more patient treatments.

17. The devices of claim 15, wherein melanin in the hair follicle is the chromophore for the skin rejuvenation and said treatments wherein said laser device or other intense pulsed light between 100 to 3000 nm is appropriate for targeting melanin in the skin.

18. The devices of claim 15 wherein said pulse length for laser light from said laser device is from about 1 nanosecond to about 1 second, and more preferably wherein said pulse length is from about 1 nanoseconds to about 300 milliseconds.

19. The devices according to claim 15, wherein the laser or equivalent light energy fluence received at said skin tissue is 300 J/cm² or less, preferably greater than 1 J/cm² and further comprises cooling said tissue and most preferably from about 1×10⁻⁶ J/cm² to 1 J/cm².

20. The devices of claim 15, further comprising cooling said tissue to maintain a temperature of said tissue below a threshold for thermal injury including a step of maintaining the temperature of said tissue at or below 38 degrees Celsius.
